(19) United States
(12) Patent Application Publication
(10) Pub. No.: US 2002/0098488 A1
(43) Pub. Date: Jul. 25, 2002

(54) ATM mutations in breast cancer

(76) Inventors: Shlomit Gilad, Gedera (IL); Rami Skaliter, Nes Ziona (IL)

Correspondence Address: John P. White, Cooper & Dunham LLP, 1185 Avenue of the Americas, New York, NY, 10036, US

(21) Appl. No.: 09/810,993

(22) Filed: Mar. 16, 2001

Related U.S. Application Data
(60) Provisional application No. 60/189,761, filed on Mar. 16, 2000.

(51) Int. Cl. C12Q 1/68

(52) U.S. Cl. 435/6

(57) ABSTRACT

According to the present invention, there is provided a method of testing a subject to determine if the subject has a predisposition for developing primary or bilateral breast cancer which includes the steps of detecting a mutation in the open reading frame of the ATM gene (SEQ.ID.NO: 1) in a cDNA sample from the subject, in a genomic DNA sample from the subject, which mutation is selected from the group consisting of the mutations set forth in Table 4 and Table 5; or detecting a mutation in the mRNA corresponding to the open reading frame of the ATM gene (SEQ.ID.NO: 1) in a mRNA sample from the subject, which mutation is selected from the group consisting essentially of RNA complementary to the mutations set forth in Table 4 and Table 5, wherein the presence of such a mutation indicates that the subject has a predisposition for developing primary or bilateral breast cancer. Also provided is an isolated cDNA having a nucleotide sequence which differs from the sequence set forth in SEQ.ID.NO: 1 by including a mutation selected from the group consisting essentially of mutations in position 378 T→A, position 3383 A→G, position 1636 C→G, position 2614 C→T, position 6437 G→C, position 2932 T→C, position 2289 T→A, position 6096 A→T, position 6176 C→T, position 6919 C→T, position 3925 G→A, position 6067 G→A, position 2119 T→C, position 1810 C→T, and position 4388 T→G. A marker for determining a predisposition for breast cancer is also provided.

### CROSS-REFERENCE TO RELATED APPLICATIONS

This application claims the benefit of priority under 35 U.S.C. Section 119(e) of U.S. Provisional Patent Application No. 60/189,761 filed Mar. 16, 2000, which is incorporated herein by reference.

### BACKGROUND OF THE INVENTION

1. Field of the Invention

The present invention generally relates to the relationship of ATM mutations and breast cancer. More specifically, the present invention relates to the use of this relationship in detecting cancer prior to large tumor growth.

2. Description of Related Art

Ataxia-telangiectasia (A-T) is a pleiotropic inherited disease characterized by neurodegeneration, cancer, immunodeficiencies, radiation sensitivity, and genetic instability. The gene responsible for A-T is called ATM, discovered by Shiloh et al. in 1995 (Savitsky, K. et al., 1995). The ATM gene extends over 150 kb of genomic DNA (Uziel, T. et al., 1996) and is transcribed into a large transcript of about 13 kb, representing 66 exons (Uziel, T. et al., 1996, Savitsky, K. et al., 1995, Savitsky, K. et al., 1997). The open reading frame of this transcript predicts a 370 kDa protein composed of 3,056 amino acids. The ATM product is homologous to several cell cycle checkpoint proteins from other organisms and is thought to play a crucial role in a signal transduction network that modulates cell cycle checkpoints, genetic recombination, apoptosis and other cellular responses to DNA damage (Meyn. M. S., 1999).

A-T cells respond abnormally to radiation-induced DNA damage and are remarkably sensitive to ionizing radiation. M. Swift and others (Morrell, et al., 1990, Swift, M., et al., 1987, Swift, M., et al., 1991, Easton, D. F., 1994) have suggested that exposure to radiation may predispose A-T carriers to the development of cancer more than non-carriers (Morrell, et al., 1990, Swift, M., et al., 1987, Swift, M., et al., 1991, Easton, D. F., 1994). Studies of relatives of A-T patients have provided consistent support for increased risk of breast cancer in female A-T heterozygotes. (Meyn, M. S., 1999). Although A-T homozygotes are rare, the ATM gene may thus play a role in cancer. (Morrell, et al., 1990, Swift, M., et al., 1987, Swift, M., et al., 1991, Easton, D. F., 1994).

Several studies have shown an increased risk for the development of breast cancer in women who had previously been treated with radiotherapy for Hodgkin's Disease (HD) (Hancock, et al., 1993, Yahalom, J. et al., 1992, Aisenberg, A. C. et al., 1997). It would therefore be useful to determine whether germline (inherited) sequence variations in ATM influence: 1. Breast cancer risk; 2. Bilateral breast cancer risk and 3. Response to radiation therapy (radiosensitivity).

### SUMMARY OF THE INVENTION

According to the present invention, there is provided a method of testing a subject to determine if the subject has a predisposition for developing primary or bilateral breast cancer which includes the steps of detecting a mutation in the open reading frame of the ATM gene (SEQ.ID.NO: 1) in a cDNA sample from the subject, in a genomic DNA sample from the subject, which mutation is selected from the group consisting of the mutations set forth in Table 4 and Table 5; or detecting a mutation in the mRNA corresponding to the open reading frame of the ATM gene (SEQ.ID.NO: 1) in a mRNA sample from the subject, which mutation is selected from the group consisting essentially of RNA complementary to the mutations set forth in Table 4 and Table 5, wherein the presence of such a mutation indicates that the subject has a predisposition for developing primary or bilateral breast cancer. Also provided is an isolated cDNA having a nucleotide sequence which differs from the sequence set forth in SEQ.ID.NO: 1 by including a mutation selected from the group consisting essentially of mutations in position 378 T→A, position 3383 A→G, position 1636 C→G, position 2614 C→T, position 6437 G→C, position 2932 T→C, position 2289 T→A, position 6096 A→T, position 6176 C→T, position 6919 C→T, position 3925 G→A, position 6067 G→A, position 2119 T→C, position 1810 C→T, and position 4388 T→G. A marker for determining a predisposition for breast cancer is also provided.

### DESCRIPTION OF THE DRAWINGS

Other advantages of the present invention can be readily appreciated as the same becomes better understood by reference to the following detailed description when considered in connection with the accompanying drawing wherein:

FIG. 1 shows the complete open reading frame (ORF) sequence of the ATM gene (SEQ.ID. NO.1), wherein the first codon is the ATG(Met) and the last is the stop codon (TGA), and all of the designations of mutations refer to this sequence, and the entire transcript can be found under accession no. U33841.

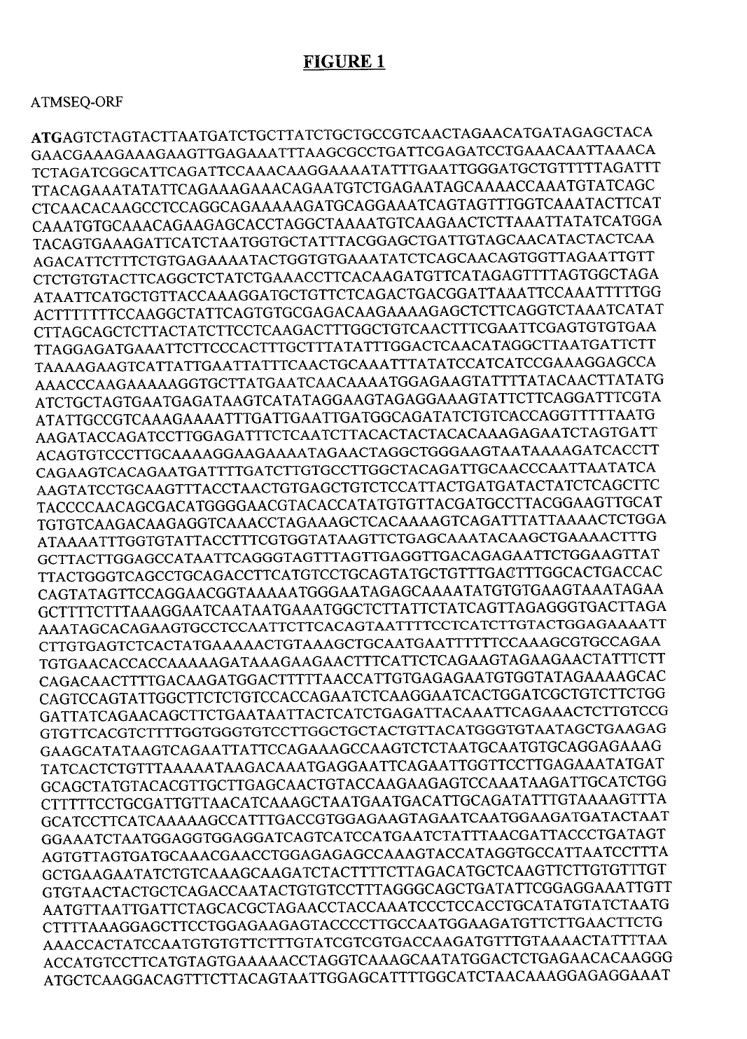

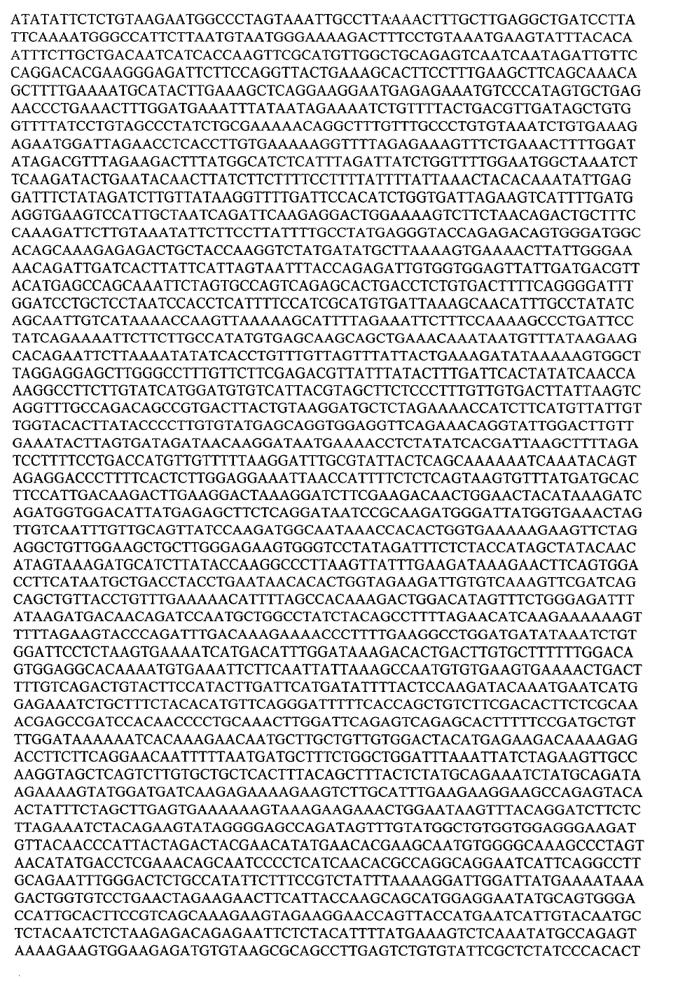

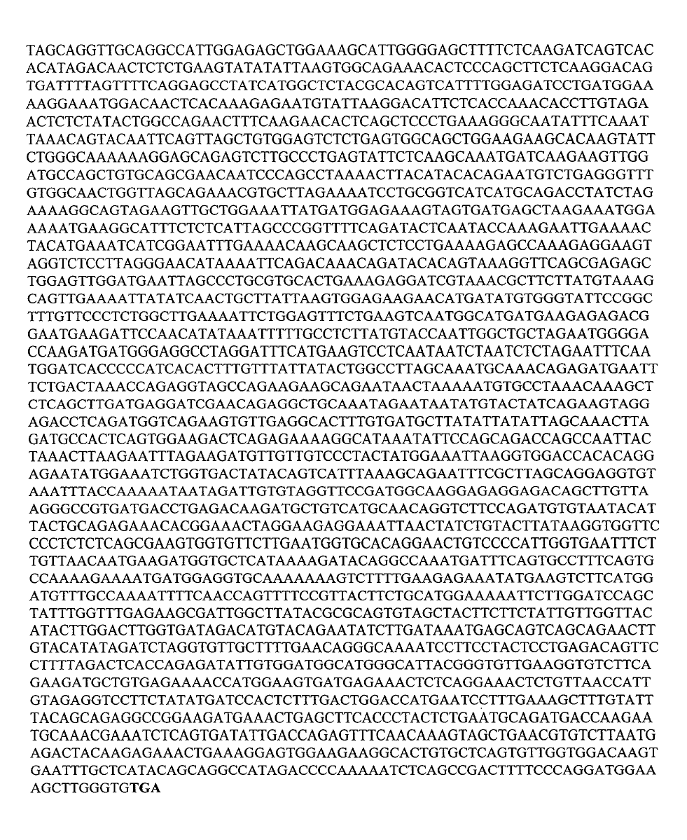

### DETAILED DESCRIPTION OF THE INVENTION

Generally, the present invention provides a method of testing a subject to determine if the subject has a predisposition for developing primary or bilateral breast cancer.

The methods of the present invention provide that either healthy women and/or women at risk (after primary breast cancer) are screened by obtaining various patient-derived materials such as tissue samples or blood (normally blood), which is then examined using methods known to those of skill in the art for the presence of the mutations. The tissue sample can include, but are not limited to, blood, mouth brush secretions, other secretions and other tissues.

The methods which are used to detect the presence of the mutations include, but are not limited to, the methods discussed below. There are many methods known to those of skill in the art for testing DNA for mutations, including point mutations. Methods which can be used for testing the mutations include methods which require the use of primers described in the specification. Mutation detection methods that are used can include, but are not limited to, polymerase chain reaction (PCR)—restriction enzyme assay (Sueoka, H. et al, 2000), PCR and LightCycler technology (Funayo, T. et al., 2000, Pais, G. et al., 2001), allele-specific PCR (MacLeod, SL et al., 2000), restriction enzyme digestion (Ho, L. L. et al., 2001), denaturing high performance liquid chromatography (dHPLC), fast and sensitive analysis of PCR-amplified DNA fragments (Oldenburg, J. et al., 2001), restriction endonuclease fingerprinting single-strand conformation polymorphism (REF-SSCP) (Jugessur, A., et al., 2000, Liu, Q. et al., 1995), and detection of single base substitutions as heteroduplex polymorphisms (White, B. M. et al., 1991).

More specifically, the method of the present invention includes the steps of detecting a mutation in the open reading frame of the ATM gene (SEQ.ID.NO:

1) in a cDNA sample from the subject, wherein the mutation is selected from the group consisting essentially of the mutations set forth in Table 4 and Table 5. The detecting step can utilize any of the above disclosed methods or any other methods known to those of skill in the art to be useful in detecting a mutation in a cDNA sample. The presence of such a mutation indicates that the subject has a predisposition for developing primary or bilateral breast cancer. detecting a mutation in the mRNA corresponding to the open reading frame of the ATM gene (SEQ.ID.NO: 1) in a mRNA sample from the subject, which mutation is selected from the group consisting essentially of RNA complementary to the mutations set forth in Table 4 and Table 5.

In another embodiment of the present invention, the method of the can include the step of detecting a mutation corresponding to a mutation in the open reading frame (ATM transcript) of the ATM gene (SEQ.ID.NO: 1) in a genomic DNA sample from the subject, wherein the mutation is selected from the group consisting essentially of the mutations set forth in Table 4 and Table 5. The detecting step can utilize any of the above disclosed methods or any other methods known to those of skill in the art to be useful in detecting a mutation in a genomic DNA sample. The presence of such mutation indicates that the subject has a predisposition for developing primary or bilateral breast cancer.

Additionally, the methods of the present invention can include the step of detecting a mutation in the mRNA, corresponding to the open reading frame of the ATM gene (SEQ.ID.NO: 1), in a mRNA sample from the subject, which mutation is selected from the group consisting essentially of RNA complementary to the mutations set forth in Table 4 and Table 5. The detecting step can utilize any of the above disclosed methods or any other methods known to those of skill in the art to be useful in detecting a mutation in a mRNA sample. The presence of such a mutation indicates that the subject has a predisposition for developing primary or bilateral breast cancer.

Also provided is an isolated cDNA having a nucleotide sequence which differs from the sequence set forth in SEQ.ID.NO: 1 by a mutation. The term “mutation” as used herein is meant to include, but is not limited to point mutations, missense, polymorphisms, and other such mutations. In the preferred embodiment, the mutation is selected from the following mutations: in position 378 T→A, position 3383 A→G, position 1636 C→G, position 2614 C→T, position 6437 G→C, position 2932 T→C, position 2289 T→A, position 6096 A→T, position 6176 C→T, position 6919 C→T, position 2442 C→A, position 3925 G→A, position 6067 G→A, position 2119 T→C, position 1810 C→T, and position 4388 T→G.

This isolated cDNA having at least one of the above mutations can also be used as a marker for determining a predisposition for breast cancer. The presence of the mutation in the cDNA is indicative of a predisposition for breast cancer. Therefore, the methods of the present invention are able to determine the presence of these mutations prior to the occurrence of cancer. The methods are also enable a determination of the whether there is a predisposition for cancer, such as breast cancer, prior to the occurrence of cancer in an individual.

The above discussion provides a factual basis for the use of the marker and method of the present invention. The methods used with and the utility of the present invention can be shown by the following non-limiting examples and accompanying figures.

### EXAMPLES

Methods:

General Methods in Molecular Biology:

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, CA (1990). In-situ (In-cell) PCR in combination with Flow Cytometry can be used for detection of cells containing specific DNA and mRNA sequences (Testoni et al, 1996, Blood 87:3822.)

### EXAMPLE 1

The current experiment was designed to determine whether germlne (inherited) sequence variations in ATM influence: 1. Breast Cancer risk; 2. Bilateral breast cancer risk and 3. Response to radiation therapy. The experiment populations were composed of three groups. 1. Contralateral breast cancer patients. (BC-BC) (with or without irradiation treatment); 2. Primary breast cancer patients. and 3. age matched healthy women.

The strategy for identification of the mutations was based on sequencing of the entire cDNA. Confirmation of the mutations was identified on the cDNA in the corresponding genomic DNA region. This full sequencing strategy is the best procedure for identifying all types of mutations and is disclosed more fully herein.

### MATERIALS AND METHODS

Total RNA Isolation from Blood Samples

Isolation of total RNA from peripheral blood was performed by Tri Reagent BD (MRC, INC), according to the manufacturer's protocol. OD verification and agarose gel electrophoresis were performed for analysis of RNA quality and quantity.

Reverse Transcription

First strand cDNA was prepared from 2 μg of total RNA. The RNA in a final volume of 5 ml was heated to 85° C. for two minutes and then kept/cooled on ice for another two minutes. A mixture comprising 2 μl of 5× Buffer (GibcoBRL), 0.5 μl of 0.5 mg/ml Oligo dT15 (Boehringer), 1 g of 0.1M DTT (GibcoBRL), 0.5 μl of 10 mM dNTP (Boehringer) and 0.5 μl of RNAsin (Promega) was added and the combination was heated to 42° C. After five minutes of incubation at 42° C., 0.5 μl of Superscript II (GibcoBRL) was added. After a further one hour of incubation at 42° C., the whole mixture was heated to 85° C. for two minutes.

ATM PCR

Amplification of ATM transcript 9355 bp was carried out with the primers ATMF and ATMR (Table 1) in a final volume of 50 μl, including 1 μl of the RT product, 1 μl of 0.1 mg/ml BSA (BioLabs), 1 μl of 25 pM of each primer, 5 μl of 10× buffer 3 (Boehringer), 2.5 μl of 10 mM dNTP (Boehringer), 0.75 μl of Expand Long Template (Boehringer) and 0.1 μl of Anti-Taq (Chimerx). The amplification was performed in the PE Cycler GeneAmp PCR 9700. The first step comprised heating at 93° C. for five minutes, followed by 20 cycles of 93° C. for 30 seconds and 68° C. for nine minutes. The third step comprised ten cycles beginning as before with 93° C. for 30 seconds and 68° C. for nine minutes, but increasing each cycle by ten seconds and completing the step with 68° C. for ten minutes.

RA and RB PCR

Two overlapping fragments, RA (4964 bp) and RB (5062 bp), were amplified using the product of the ATM RT-PCR as template (Table 1). The same mixture described above for the ATM PCR was used for PCR of each of the two fragments, respectively. The amplification was carried out under the same conditions, except for the extension time which was 3.30 minutes.

Sequencing

The RA and RB fragments were purified using QIAGEN PCR purification kit, and 200 ng of each fragments was sequenced with Big Dyes, PE ABI Prism 377, with primers as described in Table 1.

Sample Analysis

For analysis of the chromatograms, the Sequencher (Gene Code Co.) software was used.

Confirmation of Mutations

For the confirmation of each mutation, amplification of the genomic DNA was performed and the relevant region was sequenced.

Control Samples

Genomic DNA of the control samples was amplified and checked as shown in Table 2.

TABLE 1
Primers used in the study
ATM cDNA:

| | |
|---|---|
| ATMF | GTTGATACTACTTTGACCTTCCGAGTGCA |
| | GT |
| ATMR | AGGCTGAATGAAAGGGTAATTCATATACT |
| | GAAGA |

ATM RA:

| | |
|---|---|
| ATMin | GTGCAGTGAGGCATACATCAC |
| AR | CCTTCAAGTCTTGTCAATGGAAGTGCAT |

ATM RB:

| | |
|---|---|
| 2xx | GCCGTGACTTACTGTAAGGATG |
| ATMout | AAGGCTGAATGAAAGGGTAATTC |

PRIMERS FOR SEQUENCING ATM RA:

| | |
|---|---|
| LA | GTTGCTGAGATATTTCACA |
| 8P | GTTTTGGCTCCTTTCGGATGATG |
| 8X | CTTAGCAGCTCTTACTATCTTCC |
| 8K | GAAGATACCAGATCCTTGGAG |
| 6K | CTGATAATCCCAGAAGACAGCG |
| 6Q | GAGAATGTGGTATAGAAAAGCACC |
| 7out | TTCCTCTCCTTTGTTAGATGCC |
| 6in | CTAGGTCAAAGCAATATGGACTC |
| 6F | CCATAGTGCTGAGAACCCTG |
| 2A | CAGTAATAAACTAACAAACAGGTG |
| 2P | GCCATATGTGAGCAAGCAG |
| 2xx | GCCGTGACTTACTGTAAGGATG |

PRIMERS FOR SEQUENCING ATM RB:

| | |
|---|---|
| 2C | GAGGACCCTTTTCACTCTTGG |
| 1JJ | CTGGACATAGTTTCTGGGAGAT |
| 1C | GTCAGAGCACTTTTTCCGATGC |
| 3Q | CAATGTGGGGCAAAGCCCTAG |
| 3D | CAGGATTTTCTAAGCACGTTTCTG |
| 5F | CCAGAATTTTCAAGCCAGAGGG |
| 5C | CTGAGTGGCATCTAAGTTTGC |
| 4F | CCTCTTCCTAGTTTCCGTGTTTC |
| 4B | CGTGATGACCTGAGACAAGATG |
| 4A | GAGCAGTCAGCAGAACTTGTAC |

TABLE 2
Confirmation of the mutations in Genomic DNA

| | | | | Expected |
|---|---|---|---|---|
| | | | | PCR product |
| | | Primers | | size (in |
| | | for | | genomic |
| Number | Mutation | PCR | Mutation region | DNA) |
| 1 | 3161 C->G | 6in + 6B | GAGGCTGATCCTTATTCAAAA | 1.3 Kb |
| 2 | 2572 T->C | 6An + FRn | CATGAATCTATTTAACGATTA | 150 bp |
| 3 | 6235 G->A | 3Q + 3I | TATTCTTTCCGTCTATTTAAAAG | 1.5 Kb |
| 4 | 3118 A->G | 6in + 6B | CTCTGTAAGAATGGCCCTAGT | 1.3 Kb |
| 5 | 378 T->A | Uain + 8qout | ATATCATGGATACAGTGAAAG | 160 bp |
| 6 | 146 C->G | 8C + 8G | CATTCAGATTCCAAACAAGGA | 1.5 Kb |
| 7 | 5557 G->A | 1T + 1X | TTTTACTCCAAGATACAAATGAA | 2.0 Kb |
| 8 | 1636 C->G | FJ + FD | GACTTTGGCACTGACCACCAG | 190 bp |
| 9 | 2614 C->T | 6A + FB | TGCAAACGAACCTGGAGAGAG | 140 bp |

*The nucleotide number refers to the C-DNA ATM sequence. The first nucleotide of the ATG of the open reading frame was designated +1.

TABLE 2b
List of the primer sequences

| Primer | Sequence |
|---|---|
| 6in | CTAGGTCAAAGCAATATGGACTC |
| 6B | CAGCAAGAAATTGTGTAAATACTTC |
| 6An | GCCATTTGACCGTGGAGAAGTAG |
| FRn | GGTACTTTGGCTCTCTCCAGG |
| 3Q | CAATGTGGGGCAAAGCCCTAG |
| 3I | CGGAAGTGCAATGGTCCCACTG |
| Uain | GCACCTAGGCTAAAATGTCAAG |
| 8qout | ACCACTGTTGCTGAGATATTTC |
| 8C | CCTGATTCGAGATCCTGAAAC |
| 8G | GCATCTTTTTCTGCCTGGAGG |
| 1X | CCCTTTTGAAGGCCTGGATG |
| 1T | GAATCCAAGTTTGCAGGGGTT |
| Fj | GCAGTATGCTGTTTGACTTTGG |
| FD | GAAGAATTGGAGGCACTTCTGTG |
| 6A | CATTTGACCGTGGAGAAGTAGAAT |
| FB | GGTACTTTGGCTCTCTCCAGGT |

TABLE 3
ATM sequence variations in BC/BC patients

| Patient | Nucleotide | Nucleotide | | Amino-acid |
|---|---|---|---|---|
| No. | No. | substitution | Codon No. | substitution |
| #56 | 2572 | T/C | 856 | Phe → Leu |
| | 3161 | C/G | 1054 | Pro → Arg |
| #57 | 5557 | G/A | 1853 | Asp → Asn |
| | 6235 | G/A | 2079 | Val → Ile |
| #61 | 5557 | G/A | 1853 | Asp → Asn |
| | 5558 | A/T | 1853 | Asp → Val |
| #67 | 5557 | G/A | 1853 | Asp → Asn |
| #72 | 5557 | G/A | 1853 | Asp → Asn |
| #73 | 5557 | G/A | 1853 | Asp → Asn |
| | 6007 | | 2002 | Del89 |
| #75 | 3383 | A/G | 1128 | Gln → Arg |
| #80 | 2572 | T/C | 858 | Phe → Leu |
| | 3161 | C/G | 1054 | Pro → Arg |
| #83 | 5557 | G/A | 1852 | Asp → Asn |
| #90 | 5557 | G/A | 1852 | Asp → Asn |
| #93 | 1636 | C/G | 546 | Leu → Val |
| | 2614 | C/T | 872 | Pro → Ser |
| | 6995 | T/C | 2332 | Leu → Pro |
| #95 | 544 | G/C | 182 | Val → Leu |
| | 3118 | A/G | 1040 | Met → Val |
| #97 | 3161 | C/G | 1054 | Pro → Arg |
| #98 | 5557 | G/A | 1852 | Asp → Asn |
| #101 | 5557 | G/A | 1852 | Asp → Asn |
| #102 | 5557 | G/A | 1852 | Asp → Asn |
| #103 | 6235 | G/A | 2079 | Val → Ile |
| | 378 | T/A | 126 | Asp → Glu |
| #107 | 5557 | G/A | 1852 | Asp → Asn |
| | 146 | C/G | 49 | Ser → Cys |
| #112 | 6235 | G/A | 2079 | Val → Ile |
| | 378 | T/A | 126 | Asp → Glu |
| | 6437 | G/C | 2146 | Ser → Thr |
| #114 | 2932 | T/C | 978 | Ser → Pro |
| #121 | 3118 | A/G | 1040 | Met → Val |
| #122 | 3161 | C/G | 1053 | Pro → Arg |
| #124 | 146 | C/G | 49 | Ser → Cys |
| #117 | 2289 | T/A | 763 | Phe → Leu |
| #125 | 5557 | G/A | 1852 | Asp → Asn |
| #131 | 2572 | T/C | 858 | Phe → Leu |
| | 3161 | C/G | 1053 | Pro → Arg |
| #137 | 6176 | C/T | 2059 | Thr → Ile |
| | 6096 | A/T | | Arg → Ser |
| #138 | 4258 | C/T | 1420 | Lue → Phe |
| | 2119 | T/C | 707 | Ser → Pro |

Total: 28 carriers out of 70 patients (40%)
Carriers minus 5557: 19 carriers out of 70 patients (27%)

TABLE 4
Mutations found in the cohort of BC-BC patients.

| | | | MSKO | | MSKO | | % | % | |
|---|---|---|---|---|---|---|---|---|---|
| | | BC- | primary | Healthy | Healthy | | MSKO | Healthy | |
| No. | Mutation | BC | BC | Controls | controls | % BC-BC | pri-BC | Controls | Ref |
| 1 | 5557 G −> A | 8/70 | 18/76 | 8/63 | | 11.1% | 23.7% | 12.7% | (Sandoval, N. et |
| | | | | | | | | | al., 1999) |
| 2 | 3161 C −> G | 5/70 | 5/94 | 1/63 | 7/280 | 6.9% | 5.3% | 1.6% | (Vorechovsky, |
| | | | | | (2.5%) | | | | I. et al., 1996) |
| 3 | 2572 T −> C | 3/70 | 2/87 | 0/63 | 2/280 | 4.2% | 2.3% | 0.0% | (Vorechovsky, |
| | | | | | (0.7%) | | | | I. et al., 1996) |
| 4 | 6235 G −> A | 3/70 | 0/54 | 0/63 | 4/288 | 4.2% | 0.0% | 0.0% | (Vorechovsky, |
| | | | | | (1.4%) | | | | I. et al., 1996) |
| 5 | 3118 A −> G | 2/70 | 1/93 | 0/63 | | 2.8% | 1.1% | 0.0% | (Vorechovsky, |
| | | | | | | | | | I. et al. 1997) |
| 6 | 146 C −> G | 2/70 | 5/71 | 0/63 | | 2.8% | 7.0% | 0.0% | (Izatt L, et al. |
| | | | | | | | | | 2000) |
| 7 | 378 T −> A | 2/70 | 2/90 | 1/63 | | 2.8% | 2.2% | 1.6% | NEW |
| 8 | 5558 A −> T | 1/70 | 0/75 | 0/63 | 4/268 | 1.4% | 0.0% | 0.0% | (Sandoval, N. et |
| | | | | | (1.5%) | | | | al., 1999) |
| 9 | 3383 A −> G | 1/70 | 0/89 | 0/63 | | 1.4% | 0.0% | 0.0% | NEW |
| 10 | 1636 C −> G | 1/70 | 10/76 | 0/63 | | 1.4% | 13.2% | 0.0% | NEW |
| 11 | 2614 C −> T | 1/70 | 3/93 | 0/63 | | 1.4% | 3.2% | 0.0% | NEW |
| 12 | 544 G −> C | 1/70 | 0/64 | 0/63 | | 1.4% | 0.0% | 0.0% | (Izatt L, et al. |
| | | | | | | | | | 2000) |
| 13 | 6437 G −> C | 1/70 | 0/65 | 0/63 | | 1.4% | 0.0% | 0.0% | NEW |
| 14 | 2932 T −> C | 1/70 | 0/92 | 0/63 | | 1.4% | 0.0% | 0.0% | NEW |
| 15 | 2289 T −> A | 1/70 | 0/85 | 0/63 | 2/246 | 1.4% | 0.0% | 0.0% | NEW |
| | | | | | (0.8%) | | | | |
| 16 | 2119 T −> C | 2/70 | | 2/63 | 2/262 | 2.8% | | 3.2% | (Izatt L, et al. |
| | | | | | (0.8%) | | | | 2000) |
| 17 | 6096 A −> T | 1/70 | | 1/63 | | 1.4% | | 1.6% | NEW |
| 18 | 6176 C −> T | 1/70 | | 0/63 | | 1.4% | | 0.0% | NEW |
| 19 | 4258 C −> T | 1/70 | | 2/63 | 1/238 | 1.4% | | 3.2% | (Vorechovsky, |
| | | | | | | | | | I. et al., 1996) |

Nine of the mutations that were found in the group of BC-BC patients (Table 4) are new. The mutations that are known have not been reported, until now, to be linked to increased risk of breast cancer.

TABLE 5
Sequence variations in the healthy controls

| | | Nucleo- | | | |
|---|---|---|---|---|---|
| | Nucleo- | tide | Amino | | |
| Control | tide | substi- | Acid | Amino-acid | |
| No. | No. | tution | position | substitution | Reference |
| #2* | 6919 | C −> T | 2307 | Leu −> Phe | New |
| #6 | 5557 | G −> A | 1853 | Asp −> Asn | (Sandoval, |
| | | | | | N. et at., 1999) |
| #21 | 5557 | G −> A | 1853 | Asp −> Asn | (Sandoval, |
| | | | | | N. et al., 1999) |
| #26 | 378 | T −> A | 126 | Asp −> Glu | New |
| | 2442 | C −> A | 814 | Asp −> Glu | New |
| #29 | 6919 | C −> T | 2307 | Leu −> Phe | New |
| | 5557 | G −> A | 1853 | Asp −> Asn | (Sandoval, |
| | | | | | N. et al., 1999) |
| #36 | 3161 | C −> G | 1054 | Pro −> Arg | (Vorechovsky, |
| | | | | | I. et at 1996) and |
| | | | | | (Sandoval, N. al., |
| | | | | | 1999) |
| | 3925 | G −> A | 1309 | Ala −> Thr | New |
| | 5557 | G −> A | 1853 | Asp −> Asn | (Sandoval, |
| | | | | | N. et at., 1999) |
| #37 | 5557 | G −> A | 1853 | Asp −> Asn | (Sandoval, |
| | | | | | N. et al., 1999) |
| #40 | 5557 | G −> A | 1853 | Asp −> Asn | (Sandoval, |
| | | | | | N. et al., 1999) |
| #42 | 4258 | C −> T | 1420 | Leu −> Phe | (Vorechovsky, |
| | | | | | I. et al 1996) |
| | 6067 | G −> A | 2023 | Gly −> Arg | New |
| #46 | 2119 | T −> C | 707 | Ser −> Pro | New |
| #47 | 2119 | T −> C | 707 | Ser −> Pro | New |
| #52 | 1810 | C −> T | 604 | Pro −> Ser | New |
| | 4388 | T −> G | | Phe −> Cys | |
| #54 | 146 | C −> G | 49 | Ser −> Cys | (Vorechovsky, |
| | | | | | I. Et at 1996) |
| #55 | 6096 | A −> T | | Arg −> Ser | New |
| #57 | 4258 | C −> T | 1420 | Leu −> Phe | (Vorechovsky, |
| | | | | | I. et al 1996) |
| #61 | 5557 | G −> A | 1853 | Asp −> Asn | (Sandoval, |
| | | | | | N. et al., 1999) |
| #63 | 5557 | G −> A | 1853 | Asp −> Asn | (Sandoval, |
| | | | | | N. et al., 1999) |
| #64 | 378 | T −> A | 126 | Asp −> Glu | New |

Comments
1)12 carriers were found out of c63 controls (19%)
2)Sample #2 is the mother of an HD patient
3)Mutation at position 5557 was found 8 times

TABLE 6
Predominant sequence variations in BC-BC

| | | | Healthy | | | Amino acid |
|---|---|---|---|---|---|---|
| | | BC- | Con- | % BC- | % Healthy | change |
| No. | Mutation | BC | trols | BC | Controls | in prof |
| 1 | 3161 C −> G | 5/70 | 1/63 | 7.1% | 1.6% | Pro −> Arg |
| 2 | 2572 T −> C | 3/70 | 0/63 | 4.3% | 0.0% | Phe −> Leu |
| 3 | 6235 | 3/70 | 0/63 | 4.3% | 0.0% | Met −> Val |
| | G −> A | | | | | |
| 4 | 3118 | 2/70 | 0/63 | 2.9% | 0.0% | Val −> Ile |
| | A −> G | | | | | |
| 5 | 378 | 2/70 | 0/63 | 2.9% | 1.6% | |
| | T −> A | | | | | |

The frequency of the carriers of these mutations in BC-BC patients is 21.4%, among all the BC-BC patients. The frequency in healthy controls is 1/63=3.2%. Two combinations are unique to BC-BC: (i) position 3161(C→G)+position 2572(T→C) (3/70); and (ii) position 6235(G→A)+position 378(T→A) (2/70). Total 5/70=7% in BC-BC patients, 0/63=0% in normal healthy control.

Sixteen (16) new mutations were found, of which nine were in the cohort of patients (Table 4), and seven more were found in the healthy control cohort (Table 5). These new mutations are linked to a predisposition to cancer in males and females, particularly to breast cancer.

Total carriers among the BC-BC patients is 28/70, or 40%, whereas total carriers among healthy controls is 18/63, or 29%. Regarding the mutation at position 5557 (which is probably polymorphism), total carriers among the BC-BC patients is 14/70, or 20%, whereas total carriers among the healthy control cohort is 8/63, or 13%. Almost all (98%, corresponding to 43/44) of the sequence variations identified in this experiment were missense mutations (point mutations). This pattern is markedly different from that reported in Ataxia Telangiectasia patients, in which the predominant sequence variations lead to protein truncation.

The identified variation in the ATM sequences is distributed equally along most of its ORF, but none of the sequence variations were found within the PI-3 kinase domain in the carboxy terminal region of the gene. It is likely that mutations located on the catalytic site of the PI-3 kinase would cause severe phenotypes such as Ataxia Telangiectasia.

Mutations identified in healthy controls predominantly do not display any localization preference and all of them occur with almost an equal amount of low frequency.

Conclusion

Generally, three groups of mutations were found, which are as follows: 1) this mutation occurs predominantly in primary BC: position 146(C→G), and position 1636(C→G); 2) a similar level of occurrence exists in primary BC and BC-BC: position 378(T→A), position 2572(T→C), position 2614 (C→T), position 3118 (A→G), and position 3161(C→G); and 3) the mutation occurs predominantly in BC-BC: position 6235(G→A). The mutation at position 378(T→A) appears in BC-BC only in combination with position 6235(G→A).

There is a significant correlation between breast cancer and the specific sequence variations disclosed herein. The mutations found are significant for diagnosis of predisposition to cancer, particularly breast cancer.

### EXAMPLE 2

Screening Assays for Mutations in DNA.

This invention is directed to mutations in the ATM gene, which when found in a woman leads to a greater risk of developing primary breast cancer and/or bilateral breast cancer following primary breast cancer.

The methods of the present invention provide that either healthy women and/or women at risk are screened by obtaining various patient-derived materials such as tissue samples or blood (normally blood), which is then examined by methods known in the art for the presence of the mutations. These methods are more fully described in Example 1. Such methods include, but are not limited to, the methods discussed below. Note that there are many methods known in the art for testing genomic DNA and cDNA for mutations, including point mutations, as described in this specification. Methods which can be used for testing genomic DNA require use of the primers described in the specification. DNA Methods that are used can include, but are not limited to, the following inter alia: a. polymerase chain reaction (PCR)—restriction enzyme assay (Sueoka, H. et al, 2000); b. PCR and LightCycler technology (Funayo, T. et al., 2000, Pais, G. et al., 2001); c. allele-specific PCR (MacLeod, S L et al., 2000); d. restriction enzyme digestion (Ho, L. L. et al., 2001); e. denaturing high performance liquid chromatography (dHPLC) for fast and sensitive analysis of PCR-amplified DNA fragments (Oldenburg, J. et al., 2001); f. restriction endonuclease fingerprinting single-strand conformation polymorphism (REF-SSCP) (Jugessur, A., et al., 2000, Liu, Q. et al., 1995); and g. detection of single base substitutions as heteroduplex polymorphims (White, B. M. et al., 1991).

It is well known to those of skill in the art to screen DNA from biological samples for various genetic conditions. This has been accomplished for the following diseases inter alia: Phenylketoneuria (PKU) (Sueoka, H. et al, 2000); APRT deficiency (Funayo, T. et al., 2000); X-linked thrombocytopenia (XLT) (Ho, L. L. et al., 2001); hemophilia A (Oldenburg, J. et al., 2001);Cystic Fibrosis (CF); Gaucher's disease; Fragile-X Syndrome: and Canavan disease. Similar methods are used in the subject invention to screen women for the presence of the various mutations disclosed.

Throughout this application, various publications are referenced by author and year. Full citations for the publications are listed below. The disclosures of these publications in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of testing a subject to determine if the subject has a predisposition for developing breast cancer which comprises the steps of:
(a) detecting a mutation in the open reading frame of the ATM gene (SEQ.ID.NO: 1) in a cDNA sample from the subject, which mutation is selected from the group consisting of the mutations set forth in Table 4 and Table 5; or
(b) detecting a mutation corresponding to a mutation in the open reading frame of the ATM gene (SEQ.ID.NO: 1) in a genomic DNA sample from the subject, which mutation is selected from the group consisting of the mutations set forth in Table 4 and Table 5,
wherein the presence of such mutation indicates that the subject has a predisposition for developing breast cancer.

2. The method according to claim 1, wherein said detecting step includes detecting DNA which is characterized by including at least one mutation selected from the group consisting of mutations in position 3161 C→G, position 2572 T→C, position 6235 G→A, position 3118 A→G, position 378 T→A, position 2614 C→T, position 146 C→G, and position 1636 C→G.

3. The method according to claim 1, wherein said detecting step includes detecting DNA which is characterized by including at least two mutations selected from the group consisting of a double mutation in position 3161 (C>G) and position 2572(T>C); and a double mutation in position 6253(G>A) and position 378 (T>A).

4. A method of testing a subject, who has already developed primary breast cancer, to determine if the subject has a predisposition to develop bilateral breast cancer which comprises:
(a) detecting a mutation in the open reading frame of the ATM gene (SEQ.ID.NO: 1) in a cDNA sample from the subject a mutation, which mutation is selected from the group consisting of the mutations set forth in Table 4 and Table 5; or
(b) detecting a mutation corresponding to a mutation in the open reading frame of the ATM gene (SEQ.ID.NO: 1) in a genomic DNA sample from the subject, which mutation is selected from the group consisting of the mutations set forth in Table 4 and Table 5,
wherein the presence of such mutation indicates that the subject has a predisposition to develop bilateral breast cancer.

5. The method according to claim 4, wherein said detecting step includes detecting DNA which is characterized by including at least one mutation selected from the group selected from the group consisting essentially of mutations in position 3161 C→G, position 2572 T→C, position 6235 G→A, position 3118 A→G, position 378 T→A, position 2614 C→T, position 146 C→G, and position 1636 C→G.

6. The method according to claim 4, wherein said detecting step includes detecting DNA which is characterized by including at least two mutations selected from the group consisting of double mutation in position 3161(C>G) and position 2572(T>C); and double mutation in position 6253(G>A) and position 378 (T>A).

7. An isolated cDNA having a nucleotide sequence which differs from the sequence set forth in SEQ.ID.NO: 1 by a mutation selected from the group consisting of mutations in position 378 T→A, position 3383 A→G, position 1636 C→G, position 2614 C→T, position 6437 G→C, position 2932 T→C, position 2289 T→A, position 6096 A→T, position 6176 C→T, position 6919 C→T, position 2442 C→A, position 3925 G→A, position 6067 G→A, position 2119 T→C, position 1810 C→T, and position 4388 T→G.

8. A marker for determining a predisposition for breast cancer, wherein said marker includes a mutation in the open reading frame of the ATM gene (SEQ.ID.NO: 1).

9. The marker according to claim 8, wherein said mutation is selected from the group consisting of the mutations set forth in Table 4 and Table 5.

10. The marker according to claim 9, wherein said mutation is selected from the group consisting of mutations in position 378 T→A, position 3383 A→G, 1 o position 636 C→G, position 2614 C→T, position 6437 G→C, position 2932 T→C, position 2289 T→A, position 6096 A→T, position 6176 C→T, position 6919 C→T, position 3925 G→A, position 6067 G→A, position 2119 T→C, position 1810 C→T, and position 4388 T→G.
